(19) United States
(12) Patent Application Publication
(10) Pub. No.: US 2021/0052754 A1
(43) Pub. Date: Feb. 25, 2021

(54) AUTONOMOUS PORTABLE UNIT FOR CLEANING, DISINFECTING AND SANITISING ALL TYPES OF OBJECT, COMPRISING A LANCE INCORPORATING A STEAM GENERATOR AND A TANK OF SANITISER TO BE SPRAYED

(71) Applicants: Xavier HILAIRE, Bourg Saint Maurice (FR); Patricia FENOUILLET, Le Pontet (FR); Alain HILAIRE, Echirolles (FR)

(72) Inventor: Alain HILAIRE, Echirolles (FR)

(21) Appl. No.: 16/979,033

(22) PCT Filed: Mar. 5, 2019
(86) PCT No.: PCT/EP2019/055454

§ 371 Date: Sep. 8, 2020

(30) Foreign Application Priority Data
Mar. 7, 2018 (FR) 1851952

(51) Int. Cl. A61L 2/07; A61L 2/26

(52) U.S. Cl. CPC A61L 2/07; A61L 2202/16; A61L 2202/15; A61L 2/26

(57) ABSTRACT

A portable unit having a lance incorporating a steam generator and a sanitizer tank, which can improve the sanitizing and disinfection of surfaces with steam, and having a set of lines and power supply cable and a human-machine interface, which enables the supply of the fluids and electrical energy either in situ on any local network that has a water and power supply and a centralized air-blowing/suction device, or on a user-portable module that incorporates autonomous arrangement for managing the fluids and electricity.

### FIELD OF THE INVENTION

The present invention relates to the field of cleaning, disinfecting and sanitizing all types of objects using a spray of steam or of sanitizer.

It relates more particularly to the creation of an autonomous portable unit having a lance that incorporates within it a water-vapor (steam) generator and a tank of sanitizer to be sprayed.

### PRIOR ART

Numerous types of cleaning installation exist, in particular in the automotive sector.

In this sector, some are entirely automated and have roller brushes that clean the outer surface of vehicles. These installations are very popular since they do not require any effort on the part of the user and are relatively quick. However, these roller-brush installations use potable water drawn from public systems, this potable water also having harmful products added to it, which contaminate it. As a result, these installations exhibit increased investment and operating costs associated with the treatment of this contaminated water.

Other installations, known as self-service installations, allow the user to freely use equipment made available to them. This equipment typically comprises a high-pressure water lance, making it possible to remove dirt from the outer surface of vehicles. The water may be mixed, before leaving the lance, with a cleaning product in order to increase the cleaning effectiveness.

Self-service installations having high-pressure water lances are less expensive than automated installations. However, they are less economical in terms of potable water and electricity, are particularly contaminating and require large quantities of water in order to wash a motor vehicle. Moreover, they also require the use of additives, which contaminate the potable water used for washing, and this, just as in the case of roller-brush installations, indirectly increases their direct and indirect operating costs on account of the contamination to be combated, where this is possible.

Recently, self-service installations equipped with lances that operate using water vapor have been put into operation. The example of installations of this type that have been started up in Spain by the company OSVIC Maquinaria may be cited here.

A steam-cleaning installation has numerous advantages. In particular, it has lower investment costs than an automated installation and is more economical in terms of water than an installation that operates using high-pressure water. Moreover, the water vapor can be employed to clean both the inside and the outside of a vehicle. This is the case provided that the water vapor used is suitable for the different supports that it is to treat.

However, with the production or application process that is currently the most effective, the water vapor can bring about sometimes significant residual moisture on and in the supports treated, this being a nuisance and causing slow but certain deterioration of said supports, which recapture dust of all kinds, virtually nullifying the cleaning, while at the same time accelerating the proliferation of organic bacteria (fungi). These fungi are for their part a significant allergy source, with all the ensuing health consequences thereof.

Moreover, the drying cycles proposed are not only long but especially leave marks on the vehicles, requiring the manual use of cloths if the removal of these marks is desired.

In addition, the existing installations likewise use potable water from the public system and electricity from the local distribution grid.

In order to finish off internal cleaning, certain installations that operate with water vapor incorporate a suction system separate from the lance-cleaning station. This forces the user to carry out two separate operations at two different locations (two different stations) with two different types of equipment.

Furthermore, some of the installations that operate with water vapor also employ renewable energies by incorporating an electricity generation system such as photovoltaic panels. However, it has been found that the installations produced are notoriously inadequate at suitably and sufficiently meeting the cleaning requirements, even for a private individual working only on their own motor vehicle, primarily because of the power consumption.

However, the steam installations already in service also have major drawbacks which can be listed as follows.

First of all, these installations remain just as power intensive as the known automated or self-service installations that use high-pressure water, and they also make use of potable water drawn from the public system.

Moreover, just like the pressurized-water installations, they do not afford the possibility of adequately drying the vehicle. Thus, in general, the user has to drive the motor vehicle out of the installation wet for a period of time in order to allow the ambient air to dry the vehicle. This is a nuisance in particular because the wet vehicle can collect dust, at least partially nullifying the cleaning carried out, or because this natural drying can leave marks on the vehicle.

Furthermore, while, as mentioned above, some of these installations provide a suction system separate from the lance-cleaning station, this is not very practical for the user, who at best has to change cleaning tool while leaving their vehicle in place, or at worst has to move the latter into the vicinity of the suction system.

Finally, the starting up of the heater of a steam generator in these known installations requires a significant heating time and thus a significant waiting time for the user, and this can reduce the attractiveness of self-service steam-cleaning installations.

The Korean patent application KR 20090034133 discloses an installation for steam-cleaning a car, for cleaning both the inside and the outside of a vehicle by spraying low-pressure or high-pressure steam depending on the selection by an operator via a plurality of pressure switches. The low pressure proposed in that installation does not make it possible to obtain dry steam, thereby greatly wetting the surface in question and hence reducing the effectiveness of cleaning.

The Korean patent application KR 20100669923 discloses an installation for steam-cleaning a vehicle, said installation being supplied with water from a water cylinder and being supplied with power by a solar panel. This procedure and the features thereof are inadequate at suitably and sufficiently meeting the cleaning requirements, even for a private individual working only on their own motor vehicle.

These two patent applications have the same drawbacks as those set out above, even though the application KR 20100669923 proposes an attempt at electrical energy autonomy that can only be very partial since it is dependent on the weather conditions and has limited capacity on account of there being only one solar panel. Moreover, the use of a water cylinder is not realistic for self-service use, which needs to be designed for a large number of vehicles per day and requires the use of a water pump. It may be specified that the capacity of a water cylinder is itself insufficient for cleaning a single vehicle, whether this be just the outside or just the inside, since this type of installation, with the system presented, consumes at least between 3 and 6 liters of water for one vehicle, depending on how it is used.

Finally, none of the devices that have been proposed hitherto treat bacteria of any kind, i.e. cocci, organic or chemical bacteria.

For this reason, the inventor of the present invention has proposed and claimed in the patent application FR 3034064 A1 a novel installation for cleaning vehicles that operates in a combined manner with generation of water vapor and blowing or suction, making it possible to carry out effective cleaning both of the inside and of the outside of vehicles with a large number of conceivable cleaning combinations.

However, the cleaning installation according to that application requires the in situ installation of steam generators, and this can represent a significant operating cost in terms of energy if the installation is intended to clean objects that are smaller than vehicles or objects that are stationary by definition, and more generally any type of surface.

Thus, the inventor has sensibly proposed, in the patent application FR 3054457 A1, the incorporation of a steam generator within a portable lance, making it possible, while preserving the features and advantages of the combined and simultaneous cleaning using steam and blowing or suction of air according to the patent application FR 3034064 A1, to connect to any water system or rainwater tank, the blowing or suction of air being able to be ensured by a conventional device, and to effectively clean any object.

The inventor has now desired to increase autonomy so as to allow an individual to clean all types of objects, regardless of the accessibility thereof, either with an in situ connection to a local water system and/or power grid, or by avoiding any connection to a water system and to a power grid, at least for a sufficient period of time for a given number of cycles.

Moreover, the inventor has looked into the problems of disinfection and sanitizing that could improve the action of the steam.

The aim of the invention is to at least partially meet these needs of complete autonomy and of improving the disinfecting and sanitizing effect of the steam.

### SUMMARY OF THE INVENTION

According to a first of its aspects, the invention relates to a portable unit for cleaning, disinfecting and sanitizing all types of objects, comprising:
a cleaning lance intended to perform cleaning using both steam and the suction or blowing of air, and disinfection and sanitizing by spraying sanitizer, the lance accommodating:
a first fluidic circuit, referred to as the air-suction or air-blowing circuit, comprising a first air connection end piece designed to be connected to an air-suction and/or air-blowing device, at least one nozzle for suction or blowing of air toward the outside, a duct connecting the first air connection end piece to the air-suction or air-blowing nozzle;
a second fluidic circuit, referred to as the steam generating circuit, comprising a second air connection end piece designed to be connected to an air-blowing device, a water tank, a water atomizing chamber, designed to spray water coming from the tank in the form of droplets in an air flow coming from the second air connection end piece, means for generating droplets of steam from the droplets of spray, at least one nozzle for ejecting water vapor toward the outside, a duct connecting the second air connection end piece to the water-vapor ejection nozzle, passing through the water tank, the atomizing nozzle and the means for generating droplets of steam;
a third fluidic circuit, referred to as the sanitizer spray circuit, comprising a third air connection end piece designed to be connected to an air-blowing device, a storage tank for a sanitizer compound, at least one nozzle for atomizing and ejecting the sanitizer, designed to spray sanitizer coming from the tank in the form of droplets in an air flow coming from the third air connection end piece and then to eject the droplets of spray toward the outside, a duct connecting the third air connection end piece to the nozzle for atomizing and ejecting the sanitizer;
a power supply cable connected to the lance in order to supply electrical energy to the steam generating means;
an air-suction/blowing line connected to the first fluidic circuit;
an air-blowing line connected to the second and the third fluidic circuit;
a human-machine interface connected to a command and control unit for the air-blowing or air-suction device(s), designed to allow a user to control the latter so as to cause the lance to operate either in an air-suction or air-blowing cycle, if necessary combined with a steam spraying cycle, or in a sanitizer spraying cycle.

The term “object” should be understood herein and in the scope of the invention as being any solid of any kind. These thus include objects that are movable but also any objects that are clamped and/or immovable or inseparable from the rest. The objects preferably intended for cleaning with the aid of a lance of the portable unit of the invention are the walls of any volumes, closed or open, inside or outside, ventilated or non-ventilated.

Thus, the invention consists essentially in defining a portable unit having a lance incorporating a steam generator and a sanitizer tank, which can improve the sanitizing and disinfection of surfaces with steam, and having a set of lines and power supply cable and a human-machine interface, which enables the supply of the fluids and electrical energy either in situ on any local network that has a water and power supply and a centralized air-blowing/suction device, or on a user-portable module that incorporates autonomous means for managing the fluids and electricity.

The sanitizer may be in a liquid, semi-liquid or solid form.

The local network may be the centralized network of a building (house, apartment building, etc.) or of a means of transport (aircraft, train, motor vehicle, etc.).

As explained below, the portable module may be configured in backpack form and/or in the form of a case on wheels so as to make it easier to use the portable unit, specifically in any location that the user wishes to clean, sanitize and disinfect.

The portable unit may itself be temporarily accommodated with a plurality of other portable units in an autonomous installation for refilling the water and sanitizer tanks and for recharging the electrical storage means and discharging waste (liquid, dust, particles, etc.) into a suitable receptacle. It is thus possible to locally have a fully autonomous installation that the users of portable units can use as a station for refilling fluids, for recharging and for collecting waste.

According to one advantageous embodiment, the lance also accommodates a mass flow meter together with the microcontroller thereof, the mass flow meter being designed to measure the quantities of the air flows blown in order to atomize the water and the sanitizer compound. In this way, the quantity of air that is necessary both for atomizing the water in order to obtain droplets of steam and for atomizing the sanitizer compound is metered precisely.

The sanitizer tank may be in the form of one or more capsules inserted into the lance.

According to one advantageous embodiment, the lance comprises:
a body forming a handle accommodating the water tank and, if necessary, the microcontroller;
an active part forming a snout, accommodating the air-blowing or air-suction nozzles and the nozzles for ejecting water vapor and sanitizer;
a pivoting articulation connecting the handle to the snout, the articulation being designed to pivot the snout with respect to the handle into a plurality of angular positions, the articulation being designed to allow the ducts of the first, second and third fluidic circuits to pass through.

Advantageously, the power supply cable and the air-suction/blowing line connected to the first fluidic circuit and, respectively, the air-blowing line connected to the second and third fluidic circuits are accommodated in a single sleeve.

According to one advantageous embodiment, the portable unit according to the invention also comprises a portable module for managing the fluids and electrical energy, which is intended to be worn by the user, the portable module being connected to the lance by the lines and the power supply cable, the portable module accommodating at least:
an electrical-energy storage means for supplying the means for generating droplets of steam;
a storage tank for drawn-in waste;
the air-suction or air-blowing device connected to the first fluidic circuit;
the air-blowing device connected to the second and third fluidic circuits;
the command and control unit supplied with electrical energy by the electrical-energy storage means.

Preferably, the portable module also accommodates:
a supplementary water tank;
a water pump for supplying water to the tank of the lance from the supplementary water tank.

Also preferably, the portable module also accommodates:
a supplementary sanitizer tank;
a sanitizer pump for supplying sanitizer to the tank of the lance connected to the supplementary sanitizer tank.

With the portable module, the unit according to the invention gives the user actual autonomy, which, depending on the requirements, may be half a day or even a full day of cleaning, disinfecting and sanitizing work.

Preferably, the portable module is configured in backpack form and/or in the form of a case on wheels.

According to one advantageous variant, it is also possible to provide a belt provided with at least one holster for accommodating the lance.

According to this variant, the holster advantageously incorporates a first valve, referred to as a quick filling valve, connected to a water supply line itself connected to the supplementary water tank of the portable module, the command and control unit being configured such that when the lance is accommodated in the holster, it triggers the opening of the first quick filling valve and hence filling of the water tank of the lance from the supplementary water tank of the portable module.

Also advantageously, the holster incorporates a second quick filling valve, connected to a sanitizer supply line itself connected to the supplementary sanitizer tank of the portable module, the command and control unit being configured such that when the lance is accommodated in the holster, it triggers the opening of the second quick filling valve and hence filling of the sanitizer tank of the lance from the supplementary sanitizer tank of the portable module.

The human-machine interface may be formed by a box with buttons, of the push-button or touch-button type.

The box may have at least:
a button corresponding to an air-suction cycle;
a button corresponding to an air-blowing cycle;
a button corresponding to a combined vapor-spraying and air-blowing cycle;
a button corresponding to a combined vapor-spraying and air-suction cycle;
a button corresponding to a sanitizer-spraying cycle.

Preferably, the portable module comprises a housing connected electronically to the command and control unit, the housing being designed to accommodate a card for identification and recording of the data specific to each user. These data could be remotely monitored or down-/uploaded.

A further subject of the invention is an autonomous installation comprising:
at least one portable unit as has just been described;
a cabinet comprising:
   a compartment for stowing the portable unit;
   a recharging system for the electrical-energy storage means of the portable unit;
   a supplementary water tank with a capacity greater than that of the portable module of the portable unit;
   a waste storage tank with a capacity greater than that of the portable module of the portable unit;
   a supplementary sanitizer tank with a capacity greater than that of the portable module of the portable unit;
   another command and control unit configured to continuously measure the levels of power or fluid, respectively, in the recharging system and the water tank, waste storage tank and sanitizer-compound tank.

According to one advantageous embodiment, the command and control unit of each portable unit is configured such that, when the portable unit is stowed in the compartment, it commands the recharging of the storage means by the recharging system, the filling of the water tank of the portable module with water from the tank in the cabinet, the filling of the sanitizer tank of the portable module with sanitizer from the tank in the cabinet, and also the emptying and recovery of waste from the storage tank of the portable module to the storage tank of the cabinet.

### DETAILED DESCRIPTION

Further advantages and features of the invention will become more clearly apparent from reading the detailed description of the invention, given by way of nonlimiting illustration with reference to following figures, in which:

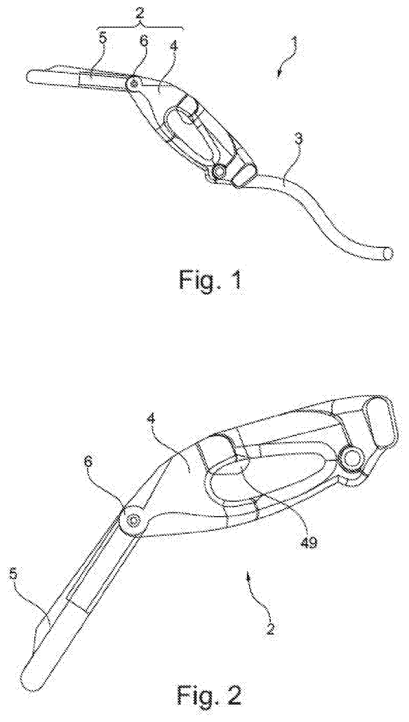

FIG. 1 is a perspective view of a portable steam-cleaning, sanitizing and disinfection unit according to one embodiment of the invention, FIG. 1 showing the components of the portable unit, namely a lance incorporating a steam generator and a sanitizer tank and a sleeve accommodating the fluid supply lines and the power supply cable;

FIG. 2 is a perspective view of the lance of the portable unit according to FIG. 1;

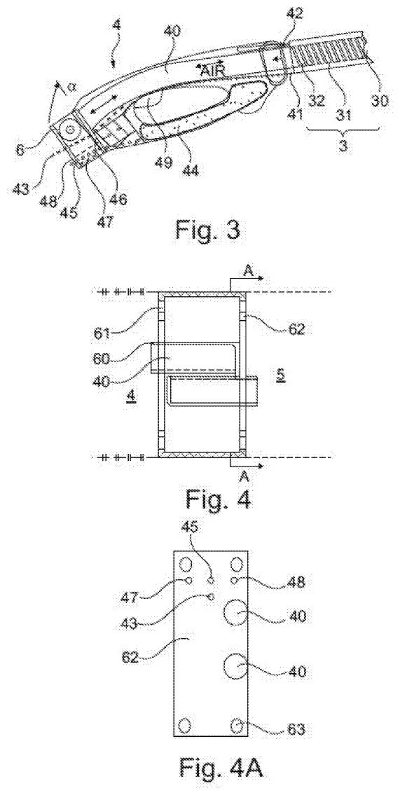

FIG. 3 is a perspective view showing hidden detail of the body forming a handle of the lance with the pivot link that forms the articulation between the snout and the body of the lance according to FIG. 2;

FIG. 4 is a schematic top view showing hidden detail of the pivot link between the body and snout of the lance;

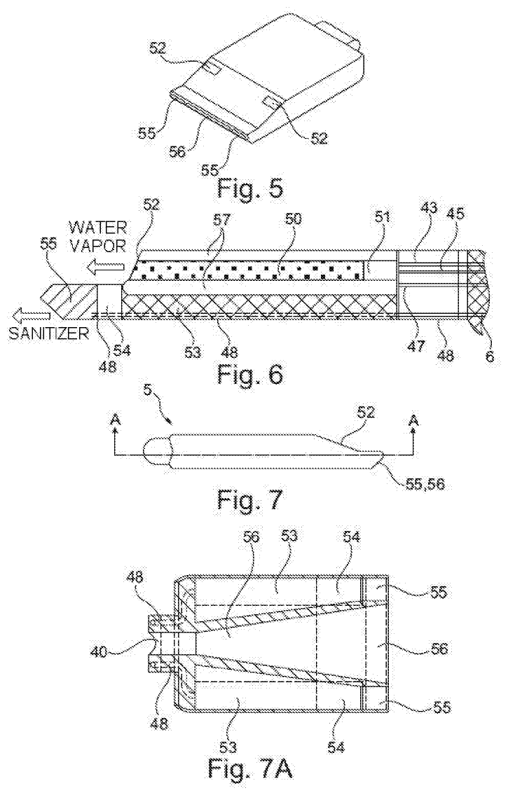

FIG. 5 is a perspective view of the snout of the lance according to FIG. 2;

FIG. 6 is a view in longitudinal section of the snout of the lance according to FIG. 2;

FIG. 7 is a side view of the snout of the lance according to FIG. 2;

FIG. 7A is a sectional view of FIG. 5 on A-A;

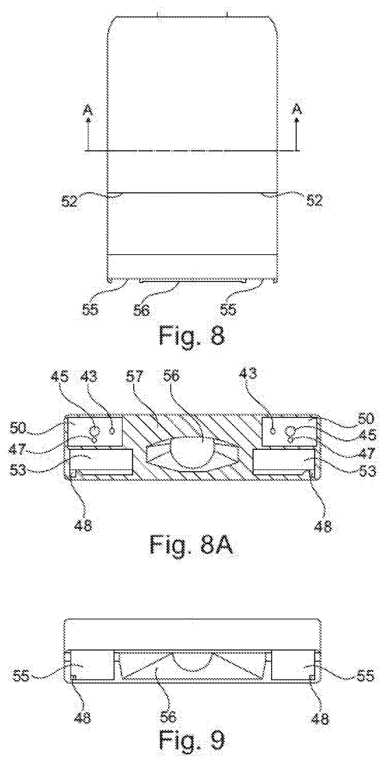

FIG. 8 is a top view of the snout of the lance according to FIG. 2;

FIG. 8A is a sectional view of FIG. 6 on A-A;

FIG. 9 is a front view of the snout of the lance according to FIG. 2;

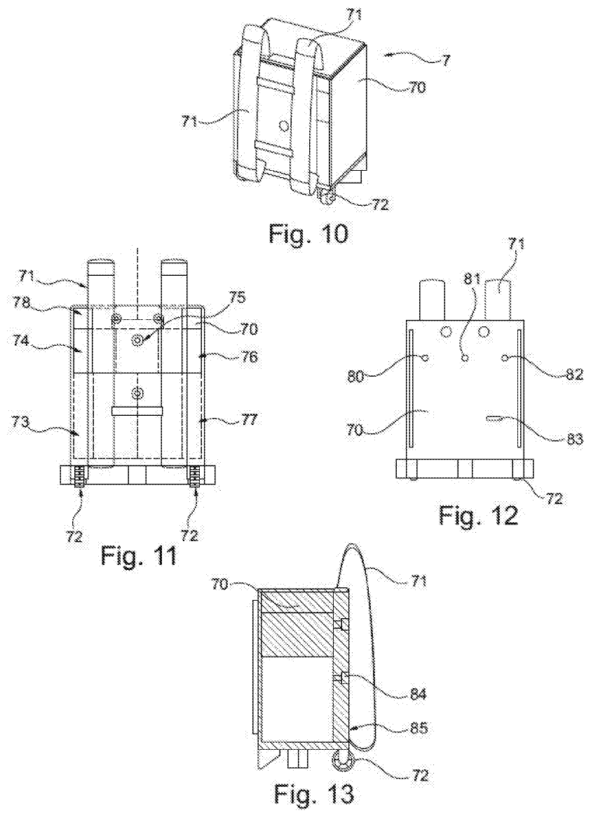

FIG. 10 is a perspective view of an example of a backpack forming a portable module for managing the fluids and electrical energy, which is intended to be worn by a user and to which the portable unit according to the invention can be connected;

FIG. 11 is a face-on view showing hidden detail of the backpack according to FIG. 10, FIG. 11 showing the different compartments and components of the backpack;

FIG. 12 is a rear view of the backpack, FIG. 12 showing the different fluid and electrical inlets and outlets for refilling the backpack with fluids and recharging it and for emptying the waste collected therein from the lance;

FIG. 13 is a view in longitudinal section of the backpack according to FIG. 10;

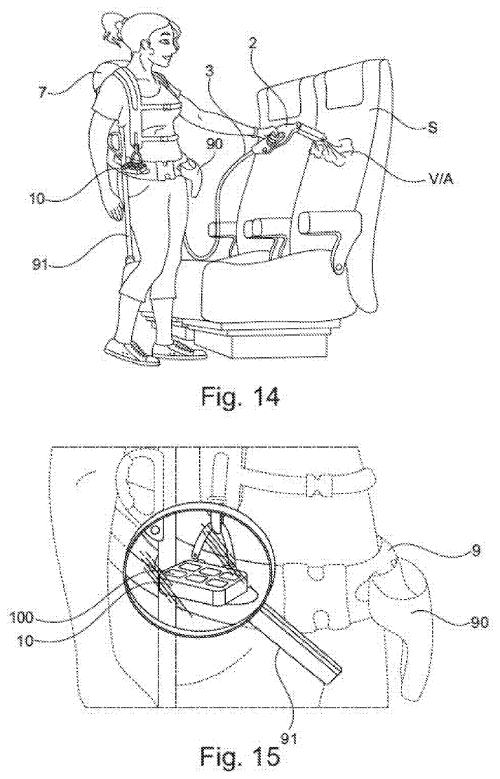

FIG. 14 schematically illustrates a user using the portable unit according to the invention for cleaning, sanitizing and disinfecting the seats of a transport vehicle, the user wearing the backpack for managing the fluids and electricity on her back;

FIG. 15 schematically illustrates a box with buttons as human-machine interface for the user in order for her to trigger the operating cycle(s) of the portable unit that she desires;

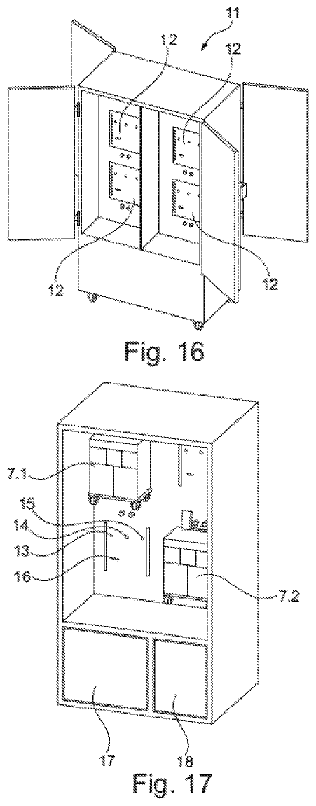

FIG. 16 is a schematic perspective view of a cabinet forming an autonomous installation for managing the fluids and electricity that is able to hold a plurality of portable units in order to individually refill them with fluid and recharge them and to unload and collect the waste that each of them has picked up;

FIG. 17 illustrates the cabinet in FIG. 16 with two portable units according to the invention accommodated therein for recharging and refilling with fluids and collecting waste.

Throughout the present application, the terms “inlet”, “outlet”, “upstream” and “downstream” should be understood with reference to the direction of circulation of the fluids (water, steam, air, or air laden with dust, sanitizer) within an autonomous installation incorporating a portable unit with a portable module according to the invention.

Similarly, the terms “top”, “bottom”, “upper” and “lower” should be considered with reference to a horizontal element.

The elements depicted are not necessarily to scale.

Not all of the fluidic and electrical circuits incorporated in the lance 2 will necessarily be described here.

FIG. 1 shows a portable unit 1 according to the invention comprising a cleaning lance 2 intended to carry out cleaning both with steam and with suction or blowing of air, and disinfection and sanitizing by spraying sanitizer, and a sleeve 3 accommodating a central air-suction/blowing line 30 and a peripheral air-blowing line 31, and also a power supply cable 32.

As illustrated schematically in FIG. 3, the peripheral line 31 and the power supply cable(s) are preferably in the form of a coil wound around the central line 30.

The lance 2 can operate according to several independent cycles as follows:

1/only suction;

2/only blowing of air for drying purposes;

3/spraying of steam combined with simultaneous suction;

4/spraying of sanitizer;

5/spraying of steam combined with simultaneous blowing of air.

In practice, cycles 3/and 4/are intended more for carrying out cleaning and sanitizing and disinfection of the interior of a passenger compartment, room of a dwelling, etc., while cycle 5/is intended more for the outside, that is to say surfaces in contact with the ambient environment.

The sleeve 3 may be advantageously formed by a flexible sheath that is resistant to twisting, squashing and other aggressions liable to be brought about during use. Typically, the sleeve may have a length of around 2 meters.

As shown in FIG. 2, the lance 2 is advantageously made up of three

parts 4, 5, one of which is the body 4 forming a handle, another of which is the snout 5, and the last of which is a pivot link 6 designed to articulate the snout 5 with respect to the body 4. This pivot link 6 thus makes it possible to incline the snout 5 at an angle α of about ±90° with respect to the body 4, thereby making it possible to position the snout as close as possible to the regions to be cleaned, sanitized and disinfected, regardless of the accessibility thereof.

The rotation of the pivot 6 can be brought about by an electromagnetic system with return springs. This rotation can be automatically blocked when a user presses the trigger 49 for commanding a lance operating cycle.

Moreover, an angular indexing system can be provided between the snout 5 and body 4, this being visible from the outside.

As shown in FIG. 3, the handle 4 is passed through by a main air-blowing and air-suction duct 40 connected directly to the central end piece of the line 30.

The end of the handle 4 comprises, respectively, a fluidic connector 41 for connecting to the peripheral air-blowing line 31 and an electrical connector 42 for connecting to the power supply cable 32.

A power supply cable 43 passes through the handle 4 in order for it to be possible to supply electrical energy to the steam generator accommodated in the snout 5.

The handle 4 incorporates a water tank 44 and supplies water to a water supply duct 45 in order to supply water to the steam generator downstream in the snout 5.

A mass flow meter 46 together with the microcontroller thereof is likewise accommodated in the handle 4. The mass flow meter 46 is supplied with air by the air line 31 from the fluidic connector.

Two air ducts 47, 48 are connected to the mass flow meter in order to supply air downstream to the water atomizing chamber 51 in the snout 5 and to the nozzles for atomizing and ejecting sanitizer 55, respectively.

Thus, the mass flow meter 46 has the function of very precisely controlling the quantity of the air flows sent through the ducts 47, 48 for downstream atomizing, in the snout 5, of the water or sanitizer. Advantageously, provision may be made to provide instructions for the flow rates of the flows of air and water, which depend, of course, on the operating cycle of the lance but also on the position of the snout (horizontal, vertical, inclined, etc.). In other words, the microcontroller can control the mass flow rate by taking account of the orientation of the snout in space, in order to maintain a uniform application of the steam or sanitizer regardless of the forces of gravity.

Finally, a trigger 49 for triggering the cycle selected beforehand by the user is mounted in a pivoting manner in the body 4.

The pivot 6 allowing the snout 5 to be articulated with respect to the handle 4 is illustrated specifically in FIGS. 4 and 4A. It comprises a flexible connector 60 accommodated between two main flanges 61, 62. The flexible connector 60 makes it possible to ensure the leaktight continuity of the main duct 40, i.e. through which the air flow is blown from the body 4 to the snout 5 and vice versa.

As can be seen in FIG. 9A, the flange 61 is held on the snout 5 via fastening means 63. This flange 61 is likewise passed through by the air ducts 47, 48 and by the water supply duct 45 for fluidically supplying the atomizing chamber and the steam generator 50 downstream and by the power supply cable 43 for supplying electrical energy to the steam generator 50. Generally, the pivot link 6 makes it possible, while ensuring any rotation from 0 to about 180° between the snout 5 and the body 4, to ensure the continuity of the air flow both for suction/blowing through the central ejection nozzle and for atomizing the water and sanitizer, and the passage of the connector technology necessary for the steam generator.

To generate the spray of water vapor, a steam generator 50 is incorporated in the top of the snout 5. This generator 50 receives upstream the droplets of water atomized in an upstream chamber 51 by the air flow in the duct 45. The generator 50 then delivers downstream the droplets of water vapor in the two ejection nozzles 52 that open out laterally at the front and the top of the snout 5 (FIG. 5). Reference may be made to the international patent application WO2018/019472A1 filed by the same applicant as the present application.

To generate the spray of sanitizer, a sanitizer tank 53 is incorporated in the bottom of the snout 5. This sanitizer tank 53 diffuses by capillary action through a suitable valve 54 in two atomizing and ejection nozzles 55 that receive upstream the air flow in the duct 48. Thus, under the effect of this air flow, the sanitizer fed into the nozzles 55 will be atomized in order to be ejected to the outside. The valve 54 is controlled electrically for the diffusion of sanitizer.

The air-blowing/suction nozzle 56 is for its part supplied directly by the central air-blowing or air-suction duct 40. In the configuration illustrated, the nozzles 55 for atomizing and ejecting the sanitizer are arranged on either side of the central nozzle 56.

As illustrated in FIG. 6, thermal insulators insulate the steam generator 50 from the outside and also insulate the sanitizer tank 53 with respect thereto.

The electrical cable 43 serves primarily to supply the steam generator 50 of the lance but also the microcontroller of the mass flow meter 46 incorporated in the handle 4 of the lance. Provision may also be made, according to a powerline communication (PLC) protocol or a wireless (Wifi, Bluetooth, etc.) protocol, for the electrical cable to exchange information with the command and control unit located remotely in particular in the backpack of the portable unit.

In order to trigger the desired operating cycle of the lance, the portable unit comprises a human-machine interface connected to the command and control unit of the air-blowing or air-suction device(s), as explained below. These air-blowing or air-suction devices, preferably formed by a turbine that is capable of operating reversibly, are directly connected to the lines of the sleeve 3.

When there is a desire for the portable unit 1 according to the invention to be able to operate in a fully autonomous manner without any connection to a local water system and a local power grid and a centralized air-blowing and suction device, provision is advantageously made to connect said portable unit 1 to a portable module 7.

As illustrated in FIGS. 10 to 13, this portable module 7 is advantageously configured in the form of a backpack.

This backpack 7 comprises an exterior envelope 70, which may be flexible or rigid and accommodates various compartments, shoulder straps 71 for making it possible to wear the backpack and advantageously rollers 72 for moving the backpack by rolling it on the ground. Provision may advantageously be made to provide the envelope with a security system for preventing any undesired opening.

More specifically, within the envelope 70, a first compartment 73 accommodates an electric battery, which therefore constitutes the source of electrical energy of the portable unit.

A second compartment 74 accommodates a water tank, which can supply the water tank 40 of the lance.

A third compartment 75 accommodates a tank for collecting waste (liquids, dust, particles) drawn in from the lance. A dust filter will advantageously be positioned in this third compartment in order that all of the particles and waste drawn in are deposited in the tank provided for this purpose.

A fourth compartment 76 accommodates a sanitizer tank, which can supply the sanitizer tank 53 of the lance.

A fifth compartment 77 accommodates at least one turbine that can operate in a reversible mode as an air-blowing and suction device connected directly to the central nozzle 50 of the lance by the central duct 41.

Finally, a sixth compartment 78 accommodates the command and control unit for electronically controlling the various components.

Fluidic connectors 80, 81, 82 are mounted in a manner passing through the exterior envelope of the backpack in order to connect the lines of the sleeve 3 to the water tank, sanitizer tank and tank for collecting waste by suction, respectively.

Likewise, a power socket 83 passing through the exterior envelope 70 makes it possible to recharge the electric battery without having to remove it from the backpack.

Finally, in order to ensure maximum comfort for the use, an air extractor 84 passes through the envelope and thermal insulation 85 is incorporated in the exterior envelope 70.

Both for reasons of comfort and automatic refilling, provision can be made to equip the portable unit 1 with a belt 9 that supports a holster 90 for the lance 5, as shown in FIG. 15. This holster 90 is intended to receive the snout of the lance 5 when it is not being used.

This holster 90 can also incorporate a first quick filling valve connected to the water tank 74 by the water supply line. Thus, when the user puts the lance 5 in the holster 90, the command and control unit triggers the opening of this first quick filling valve and hence filling of the water tank 41 of the lance.

A second quick filling valve can also be incorporated in the holster for automatically filling the sanitizer tank 53 of the lance from that of the backpack.

The belt 9 can likewise support an extension 91 that makes it possible to hold the lance 5 at a distance, for example for cleaning very high ceilings. This extension 91 is advantageously equipped with a handle provided with an on/off trigger (not shown) for starting operating cycles of the lance. This on/off trigger thus, if necessary, replaces the one on the handle of the lance.

An example of the cleaning of seats S with steam V following by a cycle of spraying sanitizer A is shown in FIG. 14.

The human-machine interface is advantageously a box 10 with buttons that is fastened to the user's belt 9, as shown in FIG. 15. Thus, depending on the desired operating cycle of the lance, the user presses one or another of the buttons 100.

It is clearly apparent from FIGS. 14 and 15 that the user can comfortably maneuver the portable unit 1 while having full autonomy in terms of fluids and electrical energy.

Provision may be made to dispose an autonomous installation in a specialized center or in any public location in order for it to be possible to refill the fluids and recharge the electrical energy of the backpacks.

As shown in FIG. 16, the autonomous installation can consist of a cabinet 11 incorporating a plurality of compartments 12 for stowing portable units 1.

More specifically, the compartments 12 are each designed to couple a backpack 7.1, 7.2 with simultaneous connection to fluidic connectors 13, 14, 15 for water, sanitizer and recovery of collected waste, respectively, and to a power socket 16 for recharging the battery of the backpack.

The cabinet is thus provided with compartments 17, 18 that accommodate both large-volume water and sanitizer tanks that can simultaneously resupply several backpacks 7.1, 7.2, . . . 7.i, and a tank for collecting the waste therefrom and a large-capacity electric battery for recharging all the batteries of the backpacks.

The cabinet can advantageously be equipped with another command and control unit configured to continuously measure the levels of power or fluid, respectively, in the recharging system and the water tank, waste storage tank and sanitizer-compound tank, and the use times and, generally, for recording and monitoring all other information and data intended to optimize the use of the installation for each of the functions thereof and for the different products associated with the functions thereof.

The invention that has just been described proposes a fully autonomous portable unit 1 that can be refilled with fluids and recharged both by means of a portable module for one individual and by means of an autonomous installation for several individuals.

The portable unit allows any user to operate the lance either according to an air-suction or air-blowing cycle, if necessary combined with a steam-spraying cycle, or according to a sanitizer-spraying cycle.

The invention is not limited to the examples that have just been described; in particular, it is possible to combine features of the examples illustrated within variants that are not illustrated.

Further variants and improvements may be contemplated without otherwise departing from the scope of the invention.

For example, while, in the examples illustrated, the lance is provided with a snout that is articulated by a pivot link to the body forming the handle, it is just as easily possible to contemplate producing a one-piece lance, i.e. one that is not articulated, which can have a straight overall shape or an overall shape with a given angle between the snout and the body.

The lance snout can be produced with several possible arrangements of the outlets of the ejection nozzles:
either with an outlet in one and the same plane for the water vapor, sanitizer and air;
or with an outlet of the steam-ejection nozzle set back with respect to the sanitizer outlet and/or the air outlet;
or with an outlet of the sanitizer-ejection nozzle set back with respect to the steam outlet and/or the air outlet.

It is also possible to contemplate equipping the lance snout with a distance detector, in particular with a laser, in order that the user is able to know in real time the distance between the outlets of the air/vapor/sanitizer-ejection nozzles and the immediately facing surface to be cleaned/disinfected/sanitized. Provision may also be made to define, for each operating cycle of the lance, a maximum distance threshold beyond which the user could be warned by an audible signal and/or a light signal and/or by vibrations that the lance snout is too far from the target surface.

It is also possible to contemplate providing the peripheral surface of the snout with a widened suction/blowing nozzle that is incorporated in the snout or is removable as required. This widened nozzle may be advantageously equipped with one or more rollers that will be designed to lift hairs from a carpet to be cleaned, disinfected or sanitized with the lance according to the invention.

In the examples explained in detail, the electrical storage means of the portable unit and of the autonomous installation are formed by batteries. Any other electrical storage means may be contemplated, such as a hydrogen storage system coupled to a fuel cell.

In order to further increase use comfort, it is also possible to incorporate loudspeakers in the human-machine interface box, which make it possible to provide the user with information about the operating cycle running.

It is likewise conceivable to send the information specific to each portable unit to a data center by a wireless communications protocol, such as Bluetooth, Wifi, etc. This information can be for example real-time data about the use time per day of a portable unit, the geographic position thereof, the levels of the tanks, power consumption, etc.

1. A portable unit for cleaning, disinfecting and sanitizing all types of objects, comprising:
a cleaning lance intended to perform cleaning using both steam and the suction or blowing of air, and disinfection and sanitizing by spraying sanitizer, the lance accommodating:
a first fluidic circuit, referred to as the air-suction or air-blowing circuit, comprising a first air connection end piece designed to be connected to an air-suction and/or air-blowing device, at least one nozzle for suction or blowing of air toward the outside, a duct connecting the first air connection end piece to the air-suction or air-blowing nozzle;
a second fluidic circuit, referred to as the steam generating circuit, comprising a second air connection end piece designed to be connected to an air-blowing device, a water tank, a water atomizing chamber, designed to spray water coming from the tank in the form of droplets in an air flow coming from the second air connection end piece, means for generating droplets of steam from the droplets of spray, at least one nozzle for ejecting water vapor toward the outside, a duct connecting the second air connection end piece to the water-vapor ejection nozzle, passing through the water tank, the atomizing nozzle and the means for generating droplets of steam;
a third fluidic circuit, referred to as the sanitizer spray circuit, comprising a third air connection end piece designed to be connected to an air-blowing device, a storage tank for a sanitizer compound, at least one nozzle for atomizing and ejecting the sanitizer, designed to spray sanitizer coming from the tank in the form of droplets in an air flow coming from the third air connection end piece and then to eject the droplets of spray toward the outside, a duct connecting the third air connection end piece to the nozzle for atomizing and ejecting the sanitizer;
a power supply cable connected to the lance in order to supply electrical energy to the steam generating means;
an air-suction/blowing line connected to the first fluidic circuit;
an air-blowing line connected to the second and the third fluidic circuit;
a human-machine interface connected to a command and control unit for the air-blowing or air-suction device(s), designed to allow a user to control the latter so as to cause the lance to operate either in an air-suction or air-blowing cycle, if necessary combined with a steam spraying cycle, or in a sanitizer spraying cycle.

2. The portable unit as claimed in claim 1, wherein the lance also accommodates a mass flow meter together with the microcontroller thereof, the mass flow meter being designed to measure the quantities of the air flows blown in order to atomize the water and the sanitizer compound.

3. The portable unit as claimed in claim 1, wherein the sanitizer storage tank is in the form of one or more capsules inserted into the lance.

4. The portable unit as claimed in claim 1, wherein the lance comprises:
a body forming a handle accommodating the water tank and;
an active part forming a snout, accommodating the air-blowing or air-suction nozzles and the nozzles for ejecting water vapor and sanitizer;
a pivoting articulation connecting the handle to the snout, the articulation being designed to pivot the snout with respect to the handle into a plurality of angular positions, the articulation being designed to allow the ducts of the first, second and third fluidic circuits to pass through.

5. The portable unit as claimed in claim 1, wherein the power supply cable and the air-suction/blowing line connected to the first fluidic circuit and, respectively, the air-blowing line connected to the second and third fluidic circuits are accommodated in a single sleeve.

6. The portable unit as claimed in claim 1, which also comprises a portable module for managing the fluids and electrical energy, which is intended to be worn by the user, the portable module being connected to the lance by the lines and the power supply cable, the portable module accommodating at least:
an electrical-energy storage means for supplying the means for generating droplets of steam;
a storage tank for drawn-in waste;
the air-suction or air-blowing device connected to the first fluidic circuit;
the air-blowing device connected to the second and third fluidic circuits;
the command and control unit supplied with electrical energy by the electrical-energy storage means.

7. The portable unit as claimed in claim 6, wherein the portable module also accommodates:
a supplementary water tank;
a water pump for supplying water to the tank of the lance from the supplementary water tank.

8. The portable unit as claimed in claim 6, wherein the portable module also accommodates:
a supplementary sanitizer tank;
a sanitizer pump for supplying sanitizer to the tank of the lance connected to the supplementary sanitizer tank.

9. The portable unit as claimed in claim 6, wherein the portable module is configured in backpack form and/or in the form of a case on wheels.

10. The portable unit as claimed in claim 1, which also comprises a belt provided with at least one holster for accommodating the lance.

11. The portable unit as claimed in claim 10, wherein the holster incorporates a first valve, referred to as a quick filling valve, connected to a water supply line itself connected to the supplementary water tank of the portable module, the command and control unit being configured such that when the lance is accommodated in the holster, it triggers the opening of the first quick filling valve and hence filling of the water tank of the lance from the supplementary water tank of the portable module.

12. The portable unit as claimed in claim 10, wherein the holster incorporates a second quick filling valve, connected to a sanitizer supply line itself connected to the supplementary sanitizer tank of the portable module, the command and control unit being configured such that when the lance is accommodated in the holster, it triggers the opening of the second quick filling valve and hence filling of the sanitizer tank of the lance from the supplementary sanitizer tank of the portable module.

13. The portable unit as claimed in claim 1, wherein the human-machine interface is formed by a box with buttons, of the push-button or touch-button type.

14. The portable unit as claimed in claim 13, wherein the box has at least:
a button corresponding to an air-suction cycle;
a button corresponding to an air-blowing cycle;
a button corresponding to a combined vapor-spraying and air-blowing cycle;
a button corresponding to a combined vapor-spraying and air-suction cycle;
a button corresponding to a sanitizer-spraying cycle.

15. The portable unit as claimed in claim 6, wherein the portable module comprises a housing connected electronically to the command and control unit, the housing being designed to accommodate a card for identification and recording of the data specific to each user.

16. An autonomous installation comprising:
at least one portable unit as claimed in claim 6;
a cabinet comprising:
a compartment for stowing the portable unit;
a recharging system for the electrical-energy storage means of the portable unit;
a supplementary water tank with a capacity greater than that of the portable module of the portable unit;
a waste storage tank with a capacity greater than that of the portable module of the portable unit;
a supplementary sanitizer tank with a capacity greater than that of the portable module of the portable unit;
another command and control unit configured to continuously measure the levels of power or fluid, respectively, in the recharging system and the water tank, waste storage tank and sanitizer-compound tank.

17. The autonomous installation as claimed in claim 16, wherein the command and control unit of each portable unit is configured such that, when the portable unit is stowed in the compartment, it commands the recharging of the storage means by the recharging system, the filling of the water tank of the portable module with water from the tank in the cabinet, the filling of the sanitizer tank of the portable module with sanitizer from the tank in the cabinet, and also the emptying and recovery of waste from the storage tank of the portable module to the storage tank of the cabinet.
